(19) United States
(12) Patent Application Publication
(10) Pub. No.: US 2007/0027064 A1
(43) Pub. Date: Feb. 1, 2007

(54) Pharmaceutical compositions for inhibiting metal ion dependent enzymatic activity and methods for the use thereof

(76) Inventor: Jerachmiel (Yori) Appelbaum, Jerusalem (IL)

Correspondence Address: Martin Moynihan;PRSTI Inc, P O Box 16446, Arlington, VA, 22215, US

(21) Appl. No.: 10/550,681

(22) PCT Filed: Mar. 25, 2004
(86) PCT No.: PCT/IL04/00279

§ 371 Date: Sep. 26, 2005

(30) Foreign Application Priority Data
Mar. 27, 2003 (IL) 155111

(51) Int. Cl. A61K 38/40; A61K 31/495; A61K 31/444; A61K 31/13

(52) U.S. Cl. 514/6; 514/332; 514/252.12; 514/423; 514/667; 514/674

(57) ABSTRACT

Methods for inhibiting metalloprotease activity; treating a pathological condition influenced by the action of MMP; treating a pathological condition influenced by cyclooxygenases, endonucleases, metallopepitidases and 5-epoxigenase; treating a pathological condition for which the presence of a metal ion is required; inhibiting the lethal factor produced by toxigenic strains of anthrax; inhibiting activities of fungi, bacteria or plants that utilize zinc-dependent methionine synthetase; inhibiting the activity of a zinc-dependent enzyme in prokaryotic systems; and inhibiting the activity of zinc-dependent enzymes; and compositions thereof, are disclosed.

### FIELD OF INVENTION

The present invention relates to pharmaceutical compositions for inhibiting the functions of metal ion-dependent enzymes, such as metalloproteases, as well as for neutralizing bacterial virulence factors by employing chelators of metals, resulting in reduction in the availability of such metals.

### BACKGROUND OF THE INVENTION

Matrix Metalloproteases

The matrix metalloproteases (MM's) constitute a multigene family of over 25 secreted and cell surface enzymes that process or degrade numerous pericellular substrates. Their targets include other proteases, proteinase inhibitors, clotting factor-binding proteins, cell surface receptors, cell-cell adhesion molecules, and virtually all structural extracellular matrix proteins. Currently, at least 19 members of this family are known Based on substrate specificity and domain organization, the MMPs can be loosely divided into four main groups: the interstitial collagenases, gelatinases, stromelysins and membrane-type MMPs (Duffy et al., Breast Can. Res. 2(4): 252-257 (2000)). MMPs are able to regulate many biological processes and, under normal homeostatic conditions, are closely regulated themselves (Sternlicht et al., Anna. Rev. Cell. Dev. Biol. 17: 463-516 (2001)).

Metalloproteases bind a metal ion such as Zn²⁺ in the active site. Some of the most widely studied metalloproteases have been digestive enzymes such as carboxypeptidases A and B and thermolysin. Traditionally, these enzymes are categorized as being either “exopeptidases,” which catalyze the step wise removal of single residues from the carboxyl terminus of a polypeptide substrate, or “endopeptidases” which catalyze the hydrolysis of non-terminal peptide bonds, especially those with hydrophobic residues.

Usually the Zn²⁺ ion serves to coordinate two to four side chains. In thermolysin and carboxypeptidase the Zn²⁺ atom is bound by interactions with the imidazole (polar rings) side chains of two His residues and with the carboxyl side chain of a Glu residue. Importantly, the Zn²⁺ is also coordinated by a water molecule, which plays a crucial role in the catalytic activity of each enzyme. Metalloproteases are known to be involved in several pathogenic processes, including Ischemia-Reperfusion injury, Neurodegeneration, Rheumatoid Arthritis and other Inflammatory diseases, toxic activity of certain bacteria and cancer metastasis and angiogenesis as well as vasculogenesis.

MMPs' Role in Cancer Metastasis and Angiogenesis

The number one cause for deaths from cancer is the late development of disseminated metastases that originate in the primary tumor. To enable this metastatic behavior, the enzymatic breakdown of the extracellular matrix (ECM) is a prerequisite and many human tumors are characterized by locally increased concentrations of MMPs (Hoekstra et al., Oncologist 6(5): 415-427 (2001)). In order to metastasize, tumor cells must intravasate, enter the circulation, extravasate, seed, and proliferate at distant sites.

Several processes in metastasis as well as normal and pathological angiogenesis require the remodeling of the extracellular matrix. MMPs comprise a class of enzymes that degrade the extracellular membrane components such as collagenase, gelatinase, and stromelysins.

Much research over the last two decades has established that angiogenesis is an essential component of tumor growth. The nutritional and respiratory needs of a tumor can be satisfied by diffusion when the tumor is small (usually less than 2 mm³). To grow larger than this, however, the tumor must first induce a blood supply to support its expansion. This is achieved by a complex multistep process of angiogenesis, which is distinct from vascularization in that the new blood vessels are not created from primitive stem cells, but rather bud off from the existing vasculature present around the tumor. The invading blood vessels insinuate into the tumor parenchyma, providing nutrition and oxygen for the growing tumor mass.

Tumor cell invasion and angiogenesis share a number of functional similarities. Like in the initiation of tumor cell invasion, the growing tips of the capillary buds secrete enzymes that digest the ECM enabling the newly forming blood vessels to burrow through the surrounding tissue. The enzymes involved in the angiogenic process seem to be similar to those required for tumor cell invasion.

One of the major constituents of the ECM is collagen, which exists in many well known subtypes, and is a prime substrate of a particular MMP, Type IV collagenase.

Type IV collagenase exists both as a 72 and as a 92 kDa molecule each of which is encoded by a unique mRNA. There is increasing evidence for a positive correlation between collagenase IV activity and tumor cell invasion. Collagenase IV is secreted in an inactive form and it seems that the plasmin generating system is involved in its activation. Inhibition of these enzymatic systems prevents malignant tumor cells from invading surrounding tissue. Agents blocking collagenase IV have been shown to prevent tumor cell invasion in vitro and in vitro. Normal cells secrete very low amounts of collagenase while invasive carcinomas secrete large amounts of this enzyme.

Collagenases are extracellular, calcium dependent, zinc endoproteinases whose substrates include the macromolecular components of extracellular matrix. Zinc is essential for their activity, while calcium is required for maximal activity. In recent years a variety of compounds have been described as showing inhibitory activity of collagenases. These include hydroxamates, carboxylates, sulfur and various phosphorus compounds, (phosphonates, phosphinates and phosphonamidates) most of them derived from peptides or peptidomimetics. However there are no reports of successful clinical application of such collagenase inhibitor compounds to date.

MMP's Role in Normal and Other Pathological Processes

MMPs are expressed during physiologic processes such as wound repair, reproduction, mammary involution, and tissue growth and remodeling. On the other hand, this class of enzymes is implicated in various diseases, syndromes and pathological processes: atherosclerosis, corneal ulceration, emphysema, osteoarthritis, osteoporosis, rheumatoid arthritis, ulcerative colitis, tumor invasion and metastasis, and ischemia-reperfusion injury to various organs. MMPs and tissue inhibitors of metalloproteinases (TIMPs) are secreted by both tumor cells and stromal cells. It is currently believed that an imbalance between active MMPs and TIMPs causes degradation of the basement membrane and allows angiogenesis, tumor growth, and invasion to occur.

MMPs' Role in Anthrax and Other Metalloprotease Toxin-Dependent Bacteria

Bacillus anthracis, a gram positive bacterium, is the causative agent of anthrax. This organism is capsulogen and toxinogenic. In addition, it is an example of a lethal infectious bacterium whose toxin is a metalloprotease. Three forms of the disease are known: gastrointestinal, skin and lung. Individuals at high risk can be vaccinated, but widespread treatment is based on administration of specific types of antibiotics that target the bacterium such as Bayer's ciprofloxacin, or Cipro. The gastrointestinal form is rare, but can cause death if left untreated. The cutaneous form is treated with penicillin injections or with oral tetracycline or erythromycin. Lung infections require intravenous penicillin. Other antibiotics also may be given and corticosteroids may also be used to reduce lung inflammation. If treatment is delayed (usually because the diagnosis isn't made promptly), then the disease may be fatal since the systemic level of the lethal toxin is already high. The problem is two-fold: first, upon proper diagnosis of the disease, the systemic level of lethal toxin may already be dangerously high, and second, the bacteria are becoming more and more resistant to antibiotics, and there is growing difficulty in finding and designing efficient drugs.

Anthrax toxin, produced by Bacillus anthraces, is composed of three proteins: protective antigen (PA), edema factor (EF), and lethal factor (LF) (Leppla, Handbook of Natural Toxins 8: 543-572 Moss et al., eds., 1995). PA alone has no toxic effect upon cells, but instead binds to specific cell surface receptors. Upon proteolytic cleavage to a 63-kDa fragment (PA63), PA forms a heptametrical membrane-inserted channel, which mediates the entry of EF and LF into the cytosol via the endosomal pathway (Gordon et al., Infect. Immun. 56: 1066-1069 (1988); Milne et al., J. Biol Chem. 269: 20607-20612 (1994)). Thus, EF or LF is toxic to cells when combined with PA.

EF is an adenylate cyclase, and together with PA forms a toxin referred to as edema toxin (Leppla, Proc. Natl. Acad. Sci. USA 79: 3162-3166 (1982)). LF and PA together form a toxin referred to as lethal toxin (LT). Lethal toxin is the dominant virulence factor produced by B. anthracis and is the major cause of death of infected animals (Pezard et al., Infect. Immun. 59: 3472-3477 (1991)). Intravenous injection of lethal toxin causes death of Fisher 344 rats in as little as 38 minutes (Ezzell et al., Infect Immun. 45: 761-767 (1984)), and incubation in vitro with mouse macrophages causes lysis in 90-120 minutes (Friedlander, J. Biol Chem. 261: 7123-7126 (1986)).

LF contains a limited sequence homology to a putative zinc-binding site at residues 686-690, HEFGH, characteristic of metalloproteases (Klimpel et al., Mol. Microbiol. 13: 1093-1100 (1994)). Substitution of the H or E residues inactivates LF (e. g., as in the recombinant LF mutant E687C) (Klimpel et al., 1994, supra) and decreases its affinity to zinc (Klimpel et al., 1994, supra; Kocki et al., FEMS Microbiol. Lett. 124: 343-348 (1994)). Certain metalloproteases inhibitors also protect macrophages against lethal toxin (Klimpel et al., 1994, supra; Menard et al., Biochem J. 320: 687-691 (1996)).

Regarding anti bacterial metalloprotease therapy, most of the treatment is directed at preventing bacterial growth and spread. Another approach may be to use specific antibodies against a toxin, to provide immunization. This approach is sometimes difficult because bacteria mutate and “lose” antigens. While not so common in gram positive bacteria, metalloproteases are very common in gram negative bacteria, including for example the Legionella pneumophila, Vibrio cholerae and others.

Nitric Oxide Enhances the Activity of MMP's

Nitric oxide (NO) is an important mediator responsible for changes in chondrocyte metabolism. Elevated levels of NO (as has been found in the cartilage and synovial fluid of arthritis patients) has been associated with decreased synthesis of aggrecan and collagen type II, and enhanced activity of matrix MMP. NO also reduces the synthesis of interleukin 1 (IL-1) receptor antagonist in chondrocytes in vitro. Expression of the inducible form of NO synthetase (iNOS) has also been detected in synoviocytes and chondrocytes from patients with inflammatory arthritis. NO itself functions directly in angiogenesis by acting on endothelial cells to “create room” for new capillaries to form.

An experimental therapeutic, COL-3 (6-demethyl-6-deoxy-4-dedimethylaminotetracycline has been shown to decrease inducible nitric oxide synthetase protein expression and nitric oxide production in rat mesangial cells. It has been suggested that nitric oxide is involved in angiogenesis. It has been shown that that endothelium-derived nitric oxide may mediate angiogenesis by supporting endothelial cell migration by an integrin-dependent mechanism. COL-3 concentrations between 2.5 and 10 g/mL inhibited nitric oxide production and inducible nitric oxide synthetase (iNOS) in murine macrophages in a concentration-dependent manner. The decrease in iNOS appears to be due to a decrease in the iNOS protein or an increase in mRNA instability rather than an effect on transcription.

Pelletier and colleagues have also demonstrated that the selective inhibition of iNOS in an experimental osteoarthritis (OA) dog model reduces the progression of cartilage lesions, as well as the production of MMP and IL-1. Therefore, they are of the opinion that that identification of agents that could reduce the activity of iNOS might form a potential new therapy in OA. Possible inhibition of iNOS has emerged as one of the modes of action of NSAIDs. They have also examined whether tetracycline might be effective against the IL-1 induced NO production in articular chondrocytes. They found that minocycline could exert its possible beneficial effects in the treatment of joint diseases via this mechanism. (J Rheumatol 2001; 28:336-40)

Therapeutic Strategies to Inhibit MMP's

Some chemotherapic treatments are already based on inhibiting MMPs. MMP inhibitors have been developed for potential therapeutic use in arthritis, cancer, periodontal disease, and corneal ulceration.

The existence of naturally occurring tissue inhibitors, such as alpha-2 macroglobulin and TIMPs 1, 2, 3 and 4, led to create man-made inhibitors, such as the AG3340 available from Agouron Pharmaceuticals, Inc, USA. This molecule is a novel inhibitor of selected MMPs, that was designed on the basis of the x-ray crystal structure of recombinant human MMPs.

Other compounds, such as Marimastat (BB-2516, British Biotech, Ltd), CGS-27023A (Novartis Pharma AG), BAY 12-9566 (Bayer Corp), D2163 (Chiroscience Group PLC), Ilomastat (GM6001, Glycomed, Inc) and COL-3 (Metastat, CMT-3, CollaGenex Pharmaceuticals, Inc) are MMP inhibitors currently in clinical trials. Golub and colleagues discovered that some tetracyclines could inhibit collagenase. Further manipulation of the tetracycline molecule resulted in the elimination of the antimicrobial properties without destroying the ability to inhibit MMPs. The resulting molecule, COL-3 (6-demethyl-6-deoxy-4-dedimethylaminotetracycline), is a highly lipophilic, chemically modified tetracycline. While COL-3 is still in phase I studies, the potential for photosensitivity may prove to be the dose-limiting toxicity and limit its clinical usefulness.

So far, the failure of MMP inhibitors to alter disease progression in metastatic cancer might have been anticipated since MMPs appear to be important in early aspects of cancer progression (local invasion and micrometastasis) and may no longer be required once metastases have been established. Current views indicate that: (1) most MMPs in tumors are made by stromal cells, not carcinoma cells; (2) cancer cells induce stromal cells to synthesize MMPs using extracellular MMP inducer (EMMPRIN) and cytokine stimulatory mechanisms; and (3) MMPs promote cell migration and the release of growth factors sequestered in the ECM (Zucker et al., Oncogene 19(56): 6642-6650 (2000)). Moreover, the paradigm of rationally designed MMP inhibitors, based on (i) the principle of “transition state analogs”, namely synthetic compounds which mimic the tetrahedral transition state of the peptide bond undergoing hydrolysis, or on (ii) the inclusion of functional groups capable of chelating the zinc present at the active site of the enzyme and essential for the performance of its action, has not been proven so far in the clinic.

MMP inhibitors are still in the developmental stage and are being studied in combination with cytotoxic agents. Nonetheless, their place in the treatment of cancer and other indications has not been clearly defined. Surrogate markers still need to be defined with this class of compounds. Many investigators believe monitoring plasma or serum levels of MMPs will not reflect the compounds' true activity in a given pathological condition. Since most MMP inhibitors are not cytotoxic, a traditional approach to drug development may prove to be futile and alternative endpoints may need to be explored.

### SUMMARY OF THE INVENTION

The background art does not teach or suggest a method to inhibit the toxic and malignant activity of metalloproteases by specifically depriving them of the essential metal ions, such as zinc (Zn²⁺) or copper (Cu²⁺) ions.

In a preferred embodiment of the invention, the use of highly specific transition metal chelators such as NNN′N′-Tetrakis-(2-pyridyl-methyl)-ethylenediamine, (TPEN) and TPEN derivatives, as therapeutic agents in pathological states where MMPs have a role, is described. The present invention presents a novel approach to the direct inhibition of metalloproteases based on the need for metal ions for activity, and indirect inhibition by (action on) abolishing NO production which solely enhance activity of matrix MMP,s. The first approach is composed of inhibiting metalloproteases by depleting them of key metal ions, or replacing them with different ions, which will render the proteases inactive, for use in treating pathological states where MMPs play a role. In another preferred embodiment of the invention, it is demonstrated that TPEN chelator prevent pathological NO activity, thus indirectly preventing MMP protein expression.

It is appreciated that TPEN, TPEN derivatives, as well as complexes formed between TPEN and inert, anti-free radical metals (such as, but not limited to, selenium, gallium, molybdenum, manganese, iron, cobalt and germanium) could be employed for use in inhibiting metal-dependent enzymatic activities. Said complexes would have the effect of inhibiting MW activity, and also act as a scavenger for free radicals, as well as SOD-mimics, in addition to the TPEN sole scavenging characteristics.

The invention is based on using specific, high affinity Zn²⁺ or Cu²⁺ chelators to cleave or replace the metal ion in the metalloprotease. The said transition metal chelator is selected from the group consisting of polyamine chelating agents, such as: ethylendiamine, diethylenetriamine, triethylenetetramine, triethylenediamine, tetraethylenep entamine, aminoethylethanol amine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine and transition metal binding peptides. The use of such chelators will not endanger the normal activity of Ca²⁺ or Mg²⁺ based enzymes, and in any case such chelators will be only employed on a limited basis, until the body has produced antibodies against the toxin, antibiotics have reduced the bacteria population or anticancer therapy has killed most of the metastatic tumor cells

By replacing the metalloprotease endogenous metal ion with a non physiological ion, such for example, Gallium, the 3D organization of the metalloprotease will be changed, and the enzyme will be rendered inactive or its affinity for its substrate will be much reduced, slowing the enzymatic process.

### BRIEF DESCRIPTION OF THE DRAWINGS

FIG. 1 shows that the toxic effect of TPEN was examined over a wide range of concentrations (0.1-10 μM) as indicated. These results show that TPEN has a toxic effect above 0.1 μM and therefore, all future experiments were carried out below that concentration;

FIGS. 2a and 2b show the results of exposing the tumor cells to various concentrations of the compound (0.0001-0.1 μM) in order to evaluate the effect of TPEN on cellular invasion. These results show a specific inhibition over two orders of magnitude of cellular invasion. The chemotactic activity of the cells was not affected by these drug concentrations.

FIG. 3 shows that the (bovine) endothelial cells that were layered on a Matrigel substrate to form capillary structures. The cultures were exposed to TPEN concentrations over two orders of magnitude (0.001-0.1 μM). The plates were analyzed using Hoffman optics. A dose dependent inhibition of capillary formation was seen in three different experiments (FIG. 3 represents one of the three assays performed).

FIGS. 4a and 4b show the efficacy of TPEN in reducing the secreted MMP-2 and MMP-9 activity, as analyzed in malignant fibrosarcoma cell line HT1080. In order to evaluate the effect of TPEN on MMP-2 and MMP-9 activities, the tumor cells were exposed to various non-toxic concentrations of the compound (0.001-0.1 micromolar) as seen in FIG. 4. After 6 and 24 hours of incubation in serum-free DMEM containing the appropriate TPEN concentrations, samples of supernatant were withdrawn and assayed for MMPs activity by zymography. These results show that TPEN inhibits MMP-2 and MMP-9 activities after 6 hours of incubation with the compound (FIG. 4A). MMP-2 activity was inhibited to a greater extent than MMP-9 activity (32% from the control MMP-2 activity and 64% from the control MMP-9 activity). This effect disappeared after 24 hours of incubation with TPEN (FIG. 4B).

FIGS. 4c and 4d are tables showing the data illustrated graphically in FIGS. 4a and 4b, respectively.

FIG. 5a shows the effect of TPEN on MMP-2 and MM-9 activity at different TPEN concentrations: 0.0001 um, 0.001 um, 0.01 um, and 0.1 um. The tables illustrated in FIGS. 5b, 5c, 5d, and 5e show data corresponding to the increasing TPEN concentrations, respectively.

### DETAILED DESCRIPTION OF THE FIGURES

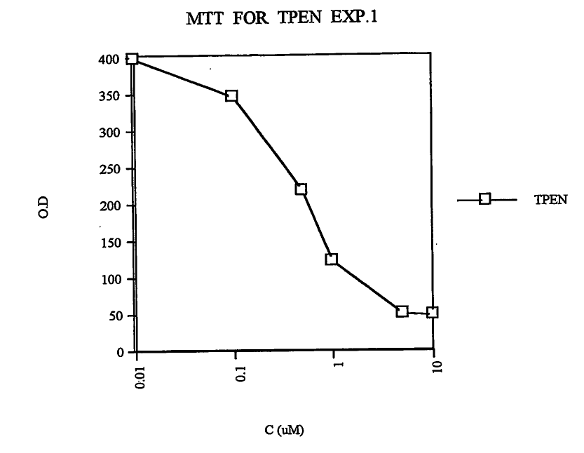

It is recognized that the induction of MMPs is a key event in angiogenesis and metastatic tumor cell invasiveness. Therefore, the chelator TPEN (NNN′N′-Tetrakis-(2-pyrydyl-methyl)-ethylenediamine) was evaluated for its ability to reduce angiogenesis and cancer cells invasiveness in in-vitro models, and a possible mechanism of reducing MPs activity was proposed. In the toxicity evaluation it was shown that TPEN has a cytotoxic effect on cancer cells above 0.1 micromolar (FIG. 1).

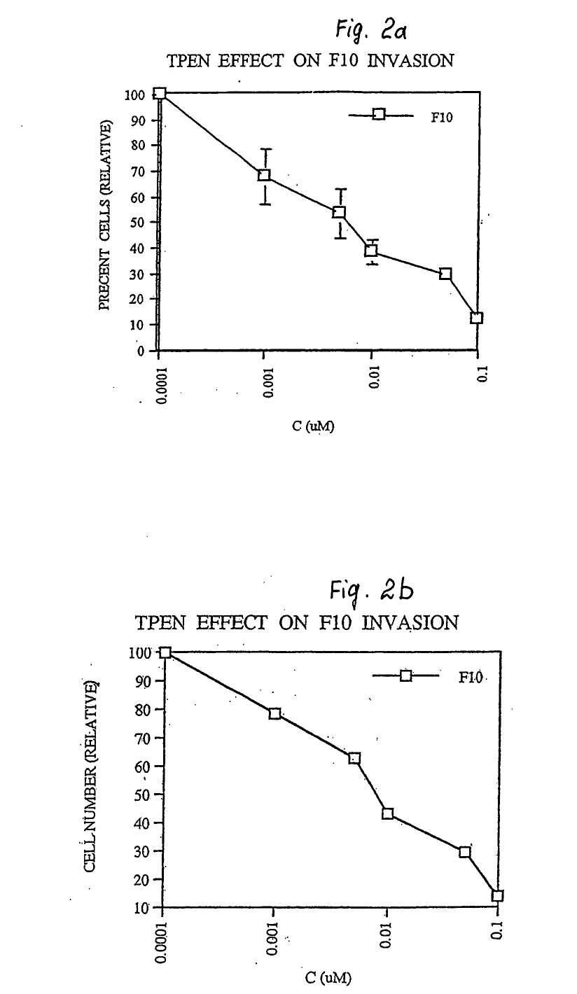

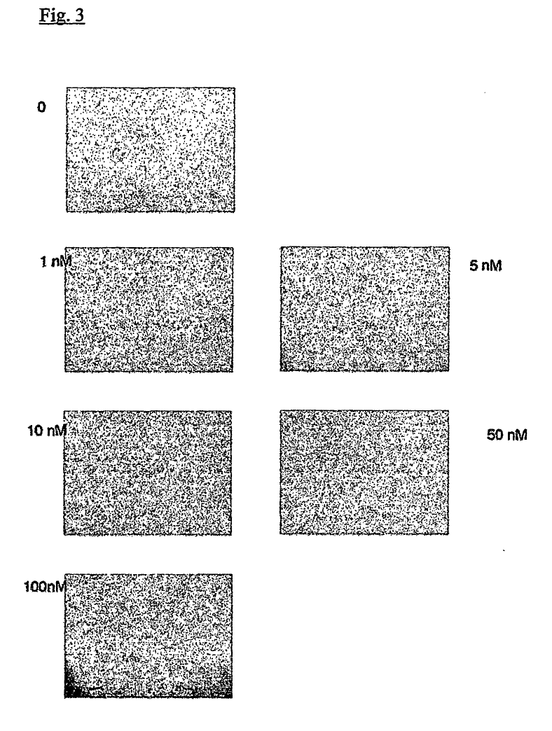

The invasive potential of a tumor depends, among many factors, on its ability to degrade basement membrane. In an in vitro model, TPEN was assessed for its ability to inhibit cancer cells invasiveness. It was shown that TPEN significantly reduces the ability of cancer cells to invade through basement membrane in a dose dependent manner (FIG. 2). The inhibition of invasiveness was not due to cell death nor to impaired cell motility, since cells were able to cross filters covered with collagen IV (which does not form a barrier to the migrating cells) Therefore, TPEN has a potential role as an anti-metastatic agent. FIGS. 2a and 2b show graphical results of two separate experiments that were carried out.

Tumor expansion beyond microscopic size is angiogenesis dependent. TPEN was assessed for its ability to inhibit angiogenesis in an in vitro model. Bovine endothelial cells assemble into capillary like structures when seeded on Matrigel (FIG. 3, 0 nM of TPEN). This ability was impaired when TPEN was added to the medium, in a concentration dependent manner (FIG. 3, 1 nM-100 nM). Therefore TPEN has a potential role as an anti-angiogenic agent. It is thus appreciated that TPEN, or other suitable high affinity metal chelators could be useful in preventing the spread of cancers of all organs, cells, and tissues of the human body. It could also have potential use in the treatment of stroke, and other central nervous system pathologies. Through the use of highly specific metal chelators, the activity of MMP's in ischemic conditions and stem cell maturation could be influenced. Any reperfusion injury or damage to any organ or system of the body could potentially be treated using MMP inhibitors such as TPEN or other suitable metal chelators. It has also been shown that inhibition of MMP prevents cyclooxygenase activity (including COX-1 and COX-2), a compound that plays a major role in brain damage and in the invasiveness of tumors. Inhibition of MMP's has also been shown to influence endonuclease, metallopepsidase, and 5-epoxigenase activities. The usage of TPEN and other chelators could have widespread ramifications in the treatment of numerous brain pathologies.

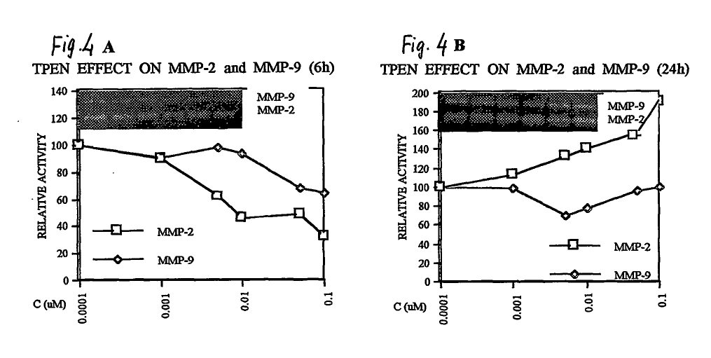

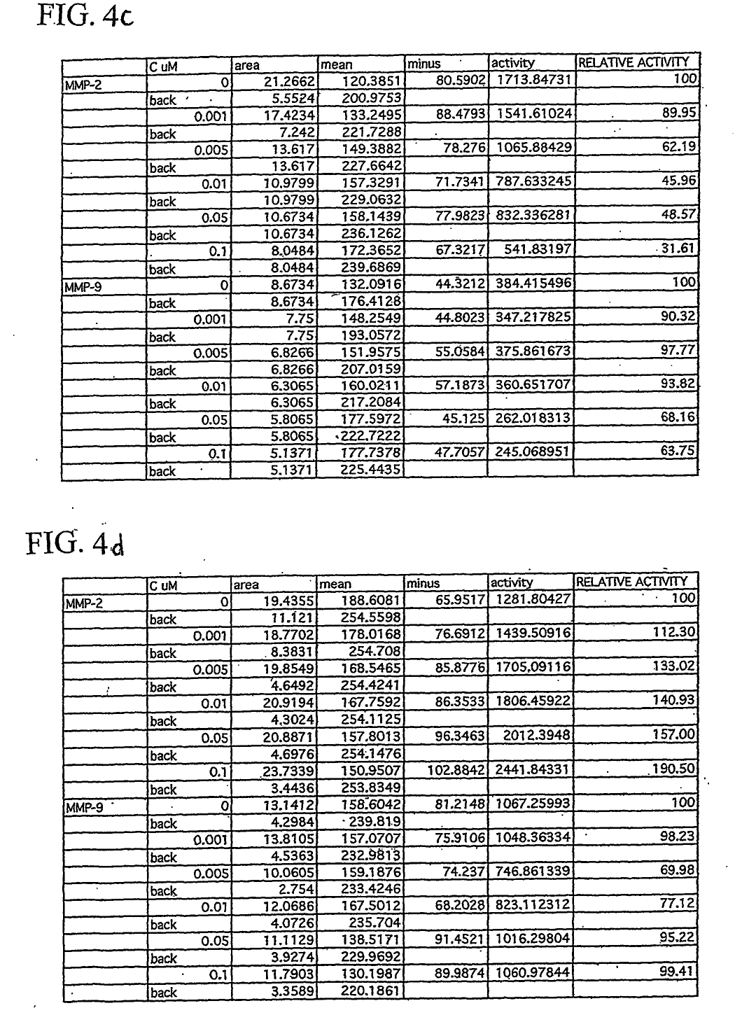

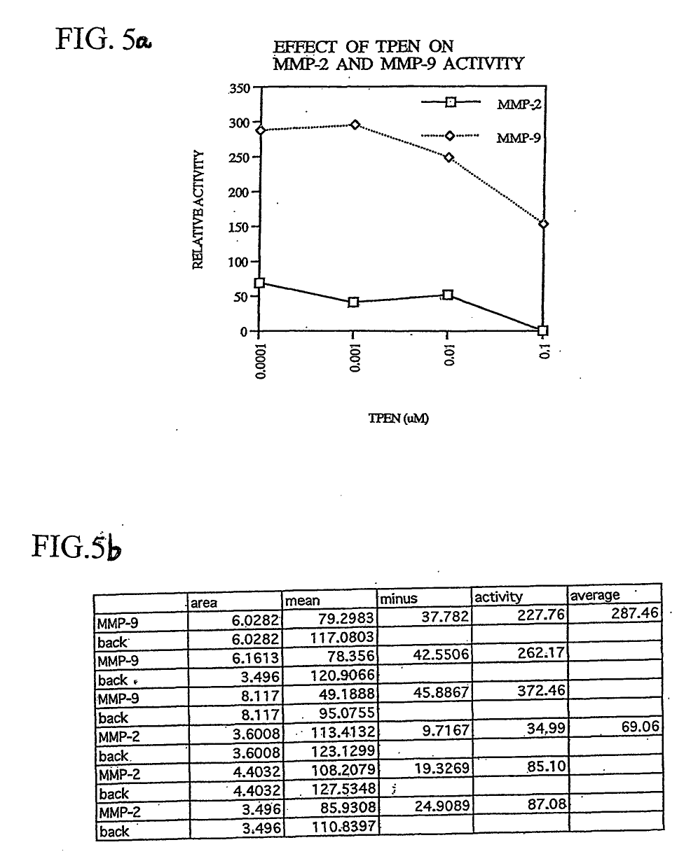

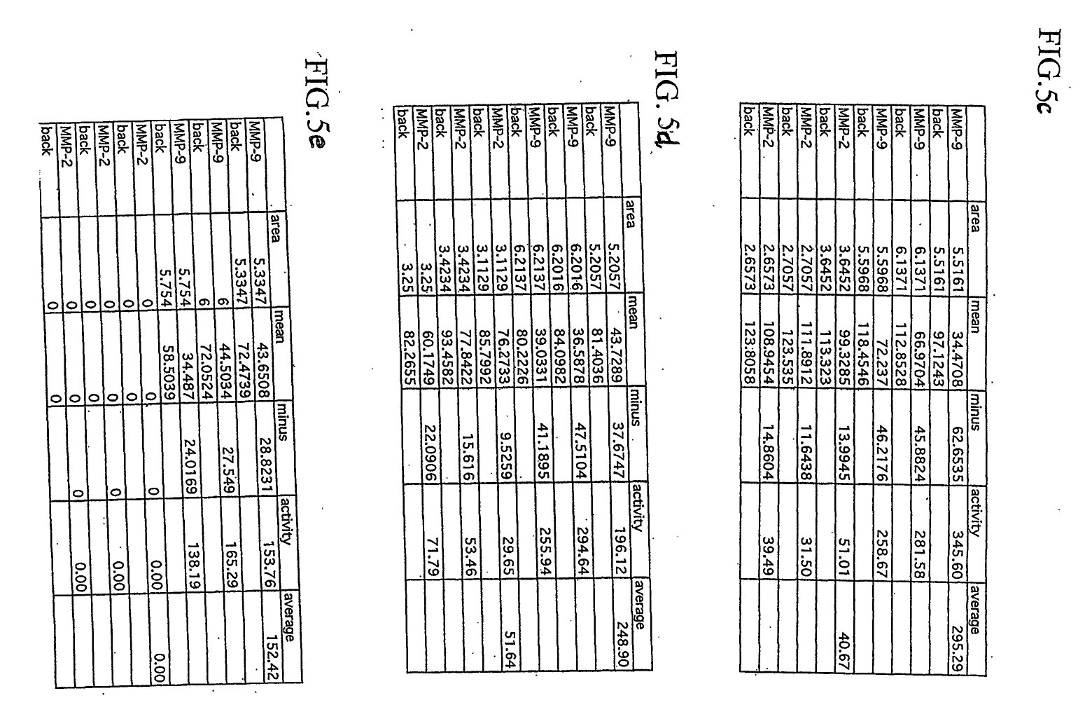

MMPs have long been associated with metastasis, and there is no doubt that there are major contributors to the metastatic process. The nature of their contribution is facilitation of the breakdown of physical barriers between primary tumor and distant sites for metastasis, and also at steps both before and after the breakdown of the apparent physical barriers. As mentioned, tumor expansion beyond microscopic size is angiogenesis dependent. MMPs play a contributory role in regulation of angiogenesis, in which there is dynamic remodeling of the ECM, and the endothelial cells display an invasive phenotype. After 6 hours of incubation, TPEN reduced the activity of secreted MMP-2 and MMP-9 in a dose dependent manner (FIG. 4A). MMP-2 activity was more sensitive to the inhibition than MMP-9 (reduction to 32% from the control MMP-2 activity and 64% from the control MMP-9 activity). The effect disappeared after 24 hours of incubation (FIG. 4B). This is with agreement with the other results, since the effect on invasiveness was evaluated after 6 hours of incubation and the anti angiogenic effect was assessed after 4 hours of incubation with TPEN. A possible explanation could be instability of TPEN in long incubations; however further study is necessary to understand the precise mechanisms of TPEN activity. The data from these experiments may be seen in FIGS. 4c (corresponding to 6 hour incubation time) and 4d (corresponding to 24 hour incubation time). Another series of experiments was carried out to show the effect of different TPEN concentrations on MMP-2 and MMP-9 activity. The results are illustrated in the graph of FIG. 5a. FIGS. 5b, 5c, 5d, and 5e show tables corresponding to the graph and to specific data resulting form TPEN concentrations of 0.0001 um (FIG. 5b), 0.001 um (FIG. 5c), 0.01 um (FIG. 5d) and 0.1 um (FIG. 5e).

These results indicate that TPEN has a specific dose dependent effect on cellular invasion and capillary formation in vitro. TPEN inhibits the activity of secreted MMP-2 and MMP-9 after 6 hours of incubation, in a dose dependent manner. Therefore, the impaired ability of the cancer cells to invade through basement membrane, and the endothelial cells to assemble into capillary-like structures was, at least in part, due to a reduced activity of MMP-2 and MMP-9 caused by TPEN.

### DETAILED DESCRIPTION OF THE PREFERRED EMBODIMENTS

In the preferred embodiment the chelator TPEN: NNN′N′-Tetrakis-(2-pyrydyl-methyl)-ethylenediamine is being used, but it is well known to those familiar with the art that the said transition metal chelator can be selected from the group consisting of polyamine chelating agents such as: ethylenediamine, diethylenetriamine, triethylenetetramine, triethylenediamine, tetraethylenepentamine, aminoethylethanol amine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicillamine and transition metal binding peptides.

Definition, structure and characteristics of TPEN:

| | |
|---|---|
| Empirical formula: | C₂₆H₂₈N₆ |
| Molecular weight: | 424.5 |
| Ion affinity: | Zn²+ > Fe²+ > Mn²+ >> Ca²+ > Mg²+ |

TPEN is a well-known heavy-metal chelator that binds zinc, iron, and copper but does not bind magnesium and calcium (under physiological blood concentration).

The selectivity of metal chelators is one of the major problems in their potential use in vivo. Most commonly used chelators bind divalent cations including calcium that is required for normal physiological function. TPEN overcomes this deficiency and moreover, it is highly lipid soluble and therefore readily permeates across cellular membranes.

In the following example, TPEN was used to inhibit cancerous cell migration across a membrane of Collagen IV in vitro. The procedure and the model are well known to those familiar with the art and is used extensively in cancer research. It provides an example to the ability of TPEN to prevent metalloprotease activity based on its Zn²⁺ chelation.

While the invention will now be described in connection with certain preferred embodiments in the following examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the mot useful and readily understood description of formulation procedures as well as of the principles and conceptual aspects of the invention.

### EXAMPLES

Material and Methods

Cell Culture

Human and murine melanoma and HUVEC cells were maintained in Minimal Essential Medium, supplemented with 5% calf serum, L-Glutamine, Sodium pyruvate, non-essential amino acids, vitamins and antibiotics (Biological Industries, Kibbutz Beth HaEmek, Israel).

### Example 1

### Analysis of Collagenase IV Activity

Sub-confluent cell cultures were incubated for 6-24 h in serum-free DMEM and the resulted supernatant were analyzed for collagenolytic activity. The collagenolytic activity was determined on a gelatin impregnated (1 mg/ml, Difco, Detroit, Mich.), SDS-PAGE 8% gel, with minor modifications. Briefly, culture media samples were separated on the substrate-impregnated gels under non reducing conditions, followed by 30 min. incubation in 2.5% Triton X-100 (BDH, England). The gels were then incubated for 16 hours at 37° C. in 50 mM Tris, 0.2M NaCl, 5 mM CaCl₂, 0.02% Brij 35 (w/v) at pH 7.6. At the end of the incubation period, the gels were stained with 0.5% Coomassie G 250 (Bio-Rad Richmond Calif.) in methanol/acetic acid/H₂O (30:10:60). The intensity of the various bands was determined on a computerized densitometer (Molecular Dynamics type 300A).

### Example 2

### Basement Membrane Invasiveness

a) Boyden chamber chemoinvasion assays were performed. Matrigel (25 mg) was dried on a polycarbonated filter (PVP free, Nucleopore). Fibroblast conditioned medium (obtained from confluent NIH-3T3 cells cultured in serum free DMEM) was used as the chemoattractant. Cells were harvested by brief exposure to 1 mM EDTA, washed with DMEM containing 0.1% bovine serum albumin and added to the Boyden chamber containing 200,000 cells. The chambers were incubated in a humidified incubator at 37° C. in 5% CO₂/95% air atmosphere for 6 h. The cells, which traversed the Matrigel layer and attached to the lower surface of the filter, were stained with Diff Quick (American Scientific Products) and counted.

### Example 3

### Chemotaxis

Chemotaxis evaluation was performed in a similar way to basement membrane invasion, with the exception that the filters were coated with 5 mg collagen IV instead of Matrigel. This amount of collagen did not form a barrier to the migrating cells but rather functioned as an attachment substratum.

### Example 4

### Tumor Growth and Metastasis in Animal Models

Experimental metastasis is being studied in a murine melanoma model. In this model, tumor cells are injected into the tail vein and the tumors which form in the lungs of the mice after 21 days are counted after appropriate fixation. Alternatively, tumor cells are injected s.c, and the growing tumors are being weighted and evaluated histologically by factor VIII for vascularization.

It is thus appreciated that TPEN may play a potential therapeutic role in any pathological condition that is influenced by the activity of metalloproteinases. Thus, TPEN may be of use as an anthrax anti-toxin. It has been shown that the cytotoxic activity of anthrax lethal factor is inhibited by metallopeptidase inhibitors (Menard, et al. Biochem J 1996 Dec 1:320 (Pt 2): 687-91). Thus, TPEN may inhibit the activity of anthrax lethal factor. It has also been shown that the lethal factor of toxigenic strains of anthrax bacteria is a Zn(2+)-endopeptidase that cleaves a group of the mitogen-activated protein kinase kinases (Vitale, et al. Biochem J 2000 Dec 15:352 Pt 3:739-45). Thus, any suitable zinc chelator could be used for inhibiting the Zn-dependent anthrax lethal factor.

It is furthermore recognized that a diverse range of enzymes use zinc as a cofactor to catalyse hydrocatalytic reactions in prokaryotic systems. Several of these systems are involved in resistance to antimicrobial agents, in virulence and pathogenesis or in metabolic pathways that are absent or markedly different in eukaryotes. Thus, there is a large potential for the use of highly specific zinc chelators for inhibiting or limiting the action of these enzymes, and for thus influencing the activity of a wide range of prokaryotic organisms.

It has also been recognized that plants, fungi, and certain bacteria cannot synthesize or transport cobalt (Vitamin B₁₂) and therefore utilize a cobalamin-independent (and zinc-dependent) methionine synthase (MetE). Since MetE has no homologue in mammals, it is a potential antibacterial/anti-fungal drug target. It is thus appreciated that suitable high affinity polyamine zinc chelators may have potential use in the inhibition of plants, fungi, and bacteria utilizing zinc-dependent MetE. By depriving the organism of essential zinc ions, the certain key metabolic activities may be inhibited. Furthermore, it is appreciated that TPEN could be useful against any bacteria that utilize metalloproteinases for vital activities, since TPEN would serve to deprive the metalloproteinases of essential metal ions.

MMP's, as mentioned previously, have been shown to play a role in many pathological states. Thus, TPEN and its derivatives, as well as other suitable metal ion chelators, could play a key role in effecting the progression of disease states. These include, but are not limited to: atherosclerosis, corneal ulceration, emphysema, osteoarthritis, osteoporosis, rheumatoid arthritis and other inflammatory disorders, ulcerative colitis, primary malignancy and tumor invasion metastasis, angiogenesis and vasculogenesis, ischemia-reperfusion injury, stroke and thrombolysis-associated hemorrhagic transformation, neurodegenerative diseases, Alzheimer's disease, Multiple Sclerosis, glaucoma, cataract and optic-nerve trauma, brain-trauma, vascular thrombolysis and restenosis, ischmic heart and lung diseases, digestive system disorders, organ rejection, infectious diseases, and mucosal pathogens such as N gonorrhoeae and P. gingivalis, and sepsis.

1. A composition for inhibiting the pathological activities of matrix metalloproteinases comprising an effective amount of NNN′N′-Tetrakis-(2-pyridyl-methyl)-ethylenediamine, (TPEN) and a pharmaceutically acceptable carrier.

2. A composition for inhibiting the pathological activities of matrix metalloproteinases as in claim 1, wherein the TPEN is in a concentration of 0.001-100 micromolar.

3. A composition for inhibiting the pathological activities of matrix metalloproteinases as in claim 1, where the active substance is a derivative, metal complexes and other forms of complexes of TPEN, including but not limited to ethylendiamine, diethylenetriamine, triethylenetetramine, triethylenediamine, tetraethylenep entamine, aminoethylethanol amine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine and transition metal binding peptides, wherein the composition is in concentration, but not limited to, of 000.1-100 micromolar.

4. A use of TPEN for the manufacture of a pharmaceutical composition for the treatment or prevention of pathological conditions influenced by the action of matrix metalloproteinases (MMPs).

5. The use, according to claim 3, wherein the disease influenced by the action of MMPs is atherosclerosis, corneal ulceration, emphysema, astma, osteoarthritis, chondrolitis and chondrosarcoma, osteoporosis, rheumatoid arthritis and other inflammatory disorders, autoimmune diseases, ulcerative colitis, primary malignancy, various types of carcinoma hodgkin's disease, various lymphomas and other hematological diseases, tumor invasion metastasis, angiogenesis and vasculogenesis, ischemia-reperfusion injury, stroke, acute MI, coronary artery diseases and thrombolysis-associated hemorrhagic transformation, neurodegenerative diseases, Alzheimer's disease, Multiple Sclerosis, glaucoma, cataract and optic-nerve trauma, brain-trauma, vascular thrombolysis and restenosis, aortic and blood vessels aneurism, types of vasculitis as Kawasaki disease, ischemic heart and lung diseases, apoptosis, diabetes, digestive system disorders, organ rejection, infectious diseases, and mucosal pathogens such as N gonorrhoeae, P. gingivalis and other periodontal diseases, and sepsis, chronic wound and granulomas.

6. The use, according to claim 1, wherein the pharmaceutical composition is formulated for oral, parenteral or intradermal administration.

7. The use according to claim 1, wherein the pharmaceutical composition is formulated as a single pharmaceutical composition.

8. A composition for inhibiting the pathological activities of matrix metalloproteinases as in claim 1, wherein the TPEN is in the form of a metal complex formed with a metal.

9. A composition according to claim 8, wherein the metal is an inert metal.

10. A composition according to claim 8, wherein the metal complex is also for use in scavenging free radicals.

11. A use of TPEN as an anti-angiogenic agent in a pharmaceutical composition.

12. A use of TPEN as an anti-metastatic agent in a pharmaceutical composition.

13. A use of TPEN as an anthrax-anti toxin.

14. A use of TPEN for inhibiting the spread of cancer in organs, cells, and tissue of the body.

15. A use of TPEN for the inhibition or prevention of ischemia and reperfusion injury in organs, cells, and tissues of the body where MMP activity plays a role.

16. A composition for inhibiting the pathological activities of matrix metalloproteinases comprising an effective amount of a highly specific metal chelator and a pharmaceutically acceptable carrier.

17. The composition, according to claim 16, wherein the metal chelator is a high affinity Zn²⁺ or Cu²⁺ polyamine chelating agent.

18. The composition, according to claim 17, wherein the polyamine chelating agent is selected from the group consisting of ethylendiamine, diethylenetriamine, triethylenetetramine, triethylenediamine, tetraethylenep entamine, aminoethylethanol amine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine and transition metal binding peptides.

19. A method for inhibiting metalloprotease activity comprising administering a pharmaceutical agent adapted for depriving metalloproteases of essential metal ions.

20. A method of treating a subject having a pathological condition influenced by the action of MMP, comprising administering to the subject an amount of TPEN or any one of its derivatives effective to treat or prevent the pathological condition.

21. A method according to claim 20, wherein the pathological condition influenced by the action of MMP is selected from the group consisting of: atherosclerosis, corneal ulceration, emphysema, astma, osteoarthritis, chondrolitis and chondrosarcoma, osteoporosis, rheumatoid arthritis and other inflammatory disorders, autoimmune diseases, ulcerative colitis, primary malignancy, various types of carcinoma hodgkin's disease, various lymphomas and other hematological diseases, tumor invasion metastasis, angiogenesis and vasculogenesis, ischemia-reperfusion injury, stroke, acute MI, coronary artery diseases and thrombolysis-associated hemorrhagic transformation, neurodegenerative diseases, Alzheimer's disease, Multiple Sclerosis, glaucoma, cataract and optic-nerve trauma, brain-trauma, vascular thrombolysis and restenosis, aortic and blood vessels aneurism ,types of vasculitis as Kawasaki disease, ischemic heart and lung diseases, apoptosis, diabetes, digestive system disorders, organ rejection, infectious diseases, and mucosal pathogens such as N gonorrhea, P. gingivalis and other periodontal diseases, and sepsis, chronic wound and granulomas.

22. A method according to claim 21, wherein the generation and/or action of MMP is induced and/or influenced by Nitros-Oxide (NO) and/or free radicals.

23. A method of treating a subject having or suspected of having a pathological condition influenced by the action of Cyclooxygenases, endonucleases, Metallopeptidases and 5-epoxygenase comprising administering to the subject an amount of TPEN effective to inhibit the action of MMP.

24. A method according to claim 22, wherein the cyclooxygenase is COX-1 or COX-2.

25. A method for treating a subject having a pathological condition for which the presence of a metal ion is required, comprising administering a metal ion chelator to the subject.

26. A method of treating a subject having or suspected of having a pathological condition influenced by the action of MMP, Cyclooxygenases, endonucleases, Metallopeptidases and 5-epoxygenase comprising administering to the subject an amount of TPEN-Germanium complex, or organic or inorganic Germaniun alone, effective to inhibit the action of MMP.

27. A method according to claim 25, wherein the pathological condition influenced by the action of MMP is selected from the group of pathological conditions consisting of: atherosclerosis, corneal ulceration, emphysema, astma, osteoarthritis, chondrolitis and chondrosarcoma, osteoporosis, rheumatoid arthritis and other inflammatory disorders, autoimmune diseases, ulcerative colitis, primary malignancy, various types of carcinoma hodgkin's disease various lymphomas and other hematological diseases, tumor invasion metastasis, angiogenesis and vasculogenesis, ischemia-reperfusion injury, stroke, acute MI, coronary artery diseases and thrombolysis-associated hemorrhagic transformation, neurodegenerative diseases, Alzheimer's disease, Multiple Sclerosis, glaucoma, cataract and optic-nerve trama, brain-trauma, vascular thrombolysis and restenosis, aortic and blood vessels aneurism types of vasculitis as Kawasaki disease, ischemic heart and lung diseases, apoptosis, diabetes, digestive system disorders, organ rejection, infectious diseases, and mucosal pathogens such as N gonorrhea, P.gingivalis and other periodontal diseases, and sepsis, chronic wound and granulomas.

28. A method according to claim 25, wherein the Germanium is in complex with a polyamine chelating agent selected from the group consisting of ethylendiaimine, diethylenetriamine, triethylenetetramine, triethylenediamine, tetraethylenep entamine, aminoethylethanol amine, aminoethylpiperazine, pentaethylenehexamine, triethylenetetramine-hydrochloride, tetraethylenepentamine-hydrochloride, pentaethylenehexamine-hydrochloride, tetraethylpentamine, captopril, penicilamine and transition metal binding peptides.

29. A method for inhibiting the lethal factor produced by toxigenic strains of anthrax bacteria comprising exposing said lethal factor to an efficient amount of a highly specific zinc chelator.

30. A method for inhibiting activities of fungi, bacteria, or plants that utilize zinc-dependent methionine synthetase (MetE) comprising delivering to an organism utilizing zinc-dependent methionine synthetase for metabolic activities an efficient amount of a highly specific zinc chelator.

31. A method for inhibiting the activity of a zinc-dependent enzyme in prokaryotic systems, comprising exposing a zinc-dependent enzyme to an efficient amount of a highly specific zinc chelator.

32. A method for inhibiting the activity of a zinc-dependent enzymes, wherein the enzymes are specifically beta-lactamases.

33. A method according to claim 30, wherein the chelator is TPEN or a TPEN derivative.
